(19) United States
(12) Patent Application Publication
(10) Pub. No.: US 2006/0116759 A1
(43) Pub. Date: Jun. 1, 2006

(54) Method of treating presbyopia and other eye conditions

(76) Inventors: Spencer P. Thornton, Nashville, TN (US); James C. Hays, Atlanta, GA (US)

Correspondence Address: Spencer P. Thornton, 5070 Villa Crest Drive, Nashville, TN, 37220, US

(21) Appl. No.: 10/999,567

(22) Filed: Nov. 30, 2004

(51) Int. Cl. A61F 2/14; A61F 9/013

(52) U.S. Cl. 623/4.1; 128/898

(57) ABSTRACT

As humans age, there is a general loss of accommodation, termed presbyopia, which eventually leaves the eye unable to focus on near objects. This loss in ability to focus on near objects is a consequence of continued growth of the lens and crowding of the lens, zonules and ciliary body within the posterior chamber. Expansion of the posterior chamber is achieved by placing radial incisions in the sclera over the ciliary body. The method of correcting presbyopia according to the present invention is by making permanent the expansion of the sclera produced by incisions made in the sclera over the ciliary muscle by means of tissue barriers placed in the incisions, preventing contraction of the incision, and acting as a retainer of the increased circumference. With expansion of the posterior chamber, the effective working distance between the lens and ciliary muscle is restored and presbyopia reversed. The tissue barriers of the present invention are solid bars containing pre-placed integral sutures for fixation. Primary open angle glaucoma and ocular hypertension can be treated by increasing the effective working range of the ciliary muscle according to the invention.

### BACKGROUND OF THE INVENTION

1) Field of the Invention

This invention relates to methods of treating presbyopia by means of surgical procedures and ophthalmic devices. This invention relates to increasing amplitude of accommodation of the eye by increasing the working space of the ciliary muscle-zonule-lens complex in the posterior chamber and allowing restoration of the hydrostatic and elastic forces within the eye. Primary open angle glaucoma and ocular hypertension are also treated by this invention.

2) Description of the Prior Art

For near vision, the effective focal length of the human eye must be adjusted to keep the image of the object focused as sharply as possible on the retina. This change in effective focal length is known as accommodation, Accommodation, or the variation of focus of the eye on objects at varying distances, is thought to result from one or more of the following: 1) Thickening of the lens with zonular relaxation resulting in increased lens power (Helmholtz 1858). 2) Ciliary muscle contraction pulls vitreous forward, forcing the lens forward, with resulting power increase (Mueller 1855). 3) Ciliary muscle contraction pulling on lens, thickening it centrally, resulting in power increase (Schachar, 1992).

The posterior chamber of the eye contains the lens, zonules and ciliary muscle. The crystalline lens is ectodermal, just as is the skin, and continues to grow throughout life. Since the growing surface of the lens cannot be shed as skin is, the lens becomes larger as we age. The sclera, on the other hand, is mesodermal, as is bone and cartilage, and matures at puberty. Thus the sclera, or outer wall of the eye, does not grow after adulthood, and gradual crowding of the posterior chamber of the eye is the result.

Presbyopia is thought to be the result of aging of the eye with concomitant crowding of the posterior chamber. Because of this crowding with aging, there is decreased room for normal function of the ciliary muscle and the lens-zonule complex, and the result is presbyopia. Because of this continued growth of the lens within a non-growing chamber, crowding of the posterior chamber occurs with aging. Any procedure or device that could expand the space in the aging posterior chamber and restore normal function of the ciliary muscle/zonule/lens complex could therefore reverse presbyopia.

All incisions, unless tightly sutured or otherwise closed, act as if tissue is added. This is noted in the eye when radial incisions are made in the periphery of the cornea, as in radial keratotomy (RK). The spread of these radial incisions causes an increase in the circumference of the cornea at right angles to the direction of the incisions and therefore increase the corneal circumference in the area of the incisions. The result is an overall flattening of the cornea and a hyperopic shift in the focal power—a treatment for myopia or nearsightedness.

Expansion of the sclera is accomplished in a manner similar to RK by placing short radial incisions in the sclera over the ciliary body. The scleral incisions weaken the sclera in the region of the ciliary body, resulting in expansion of the circumference of the eye and increasing the working distance between the ciliary body and the lens of the eye. Because this tissue is richly vascular and heals rapidly, the expansion produced by incisions may be rapidly lost without the aid of an artificial barrier to block the in-growth of new vessels and fibrosis and contraction of the incision.

One such procedure is known as anterior ciliary sclerotomy (ACS). ACS is a surgical incisional technique used to correct up to +2.00 diopters (D) of presbyopia. The procedure is based on the use of four or more radial incisions in the sclera over the ciliary body of the eye. The incisions expand the posterior chamber of the globe containing the ciliary body, zonules and crystalline lens, producing more room for the lens to accommodate for near vision. This effectively increases focal power and focal depth. The incisions are placed beginning at the surgical limbus, extending approximately 3 mm radially, stopping just anterior to the pars plana of the eye. This procedure can be enhanced by adding more incisions or reversed by suturing previously placed incisions.

One of the drawbacks of conventional ACS procedures is that the incisions rapidly heal and close, thereby reversing the beneficial effects of the procedure. Accordingly, a longer-lasting surgical method for increasing increase accommodation of the eye for near vision is needed. A barrier to prevent incision closure is therefore mandatory. Tissue barriers inserted in the radial incisions prevent closure and maintain the expansion produced by incisions.

Maintaining the expansion produced by incisions is accomplished by inserting a barrier into the incision and fixing it permanently in place, resulting in an increase in the effective working distance of the ciliary muscle/lens/zonule complex in the posterior chamber of the eye.

### SUMMARY OF THE INVENTION

A method of treating presbyopia and other eye disorders has now been found which comprises enlarging the posterior chamber and increasing the effective working distance of the ciliary muscle in the presbyopic eye.

Accordingly, it is an object of this invention to provide a method for treating presbyopia by increasing the radial distance between the lens and the ciliary muscle in the pressbyopic eye.

The present invention retains the effect of radial incisions with resulting restoration of ciliary body-zonule-lens relationships allowing forward movement of the lens on accommodation.

The method of this invention is an expansion barrier of silicone or other solid material inserted into incisions made in the sclera over the ciliary body.

This present invention consists of a specially designed rod or block of material such as silicone (in the preferred embodiment), or other solid, inert material that is placed into a radial incision made through the sclera over the ciliary muscle (ciliary body), and fixed in place. Fixation is achieved by sutures which are preplaced and integral to the invention.

This invention is unique in that it does not involve making circumferential tunnels or pockets in the sclera as in most prior art and as claimed in most of the patents cited. No implants are placed circumferentially in tunnels or pockets in the sclera. The intraocular pressure is not intentionally raised. It is not the purpose of the invention to raise intraocular pressure. No external pressure is placed on the ciliary muscle. No external force is applied on the zonules. No attempt is made, nor is there any need for shortening of the zonules. The sclera is not raised above the ciliary muscle. Expansion of the globe is circumferential over the ciliary body and is the result of stretching of the walls of the incision and the natural force of the intraocular pressure on the weakened globe wall.

The barrier, or retainer, in the preferred embodiment of the invention, is a solid rectangular rod of silicone or other inert material that is placed in the incision and permanently retained in place by sutures or other means of fixation.

According to the invention, any means of retaining expansion of the globe in the area of the ciliary muscle (ciliary body) will result in increased working space in the ciliary muscle-zonule-lens complex and allow restored accommodation. The means of weakening the sclera according to the invention is by surgical incisions but may also include chemical or laser or thermal application.

Addition of a tissue barrier or retainer according to the invention maintains the expansion of the globe produced by radial incisions over the ciliary body and results in restoration of accommodation. Glaucoma and other conditions can also be treated by increasing the scleral circumference according to the invention. With this invention the eye pressure is not increased. On the contrary, the eye pressure is lowered, and this is an additional claim of the invention.

When radial incisions are placed in the sclera, beginning at the limbus and carried 3 mm outward over the ciliary body, the scleral circumference expands (just as the cornea does in RK), producing an increase in the volume (an expansion) of the posterior chamber of the eye. The posterior chamber includes the ciliary muscle, the zonules (supporting fibrils connecting the ciliary muscle to the lens, making up the suspensory ligament of the lens) and the crystalline lens.

With increased working space the lens/zonule complex can move forward with accommodative effort, correcting the loss of this function from presbyopia. Further explanation will make this clear.

Further to understanding the importance of the invention is the fact that, though the radial incisions placed over the ciliary body produce a restoration of the relationships of the structures of the posterior chamber as in a younger eye, the rapid healing and contraction of the walls of the incisions from neovascularization and scarring cause a loss of the effect quite rapidly, with a regression of effect occurring within a few weeks. An expansion barrier prevents that contraction.

The new technology expansion barriers of this invention are inserted into the radial incisions placed over the ciliary body. Expansion of the globe circumference is produced by the radial incisions; the tissue barriers maintain that expansion.

The present invention provides a new and enhanced surgical method for the treatment of presbyopia. The Tissue Barriers and related implantation methodology have a number of advantages over approaches. Examination of the drawings and appended claims will make this clear.

### BRIEF DESCRIPTION OF THE DRAWINGS

FIG. 1. Shows an isometric view of the tissue expansion barrier of this invention.

FIG. 2. Shows an isometric view of the preferred embodiment of the tissue expansion barrier of FIG. 1 with pre-placed sutures penetrating the barrier in the upper 1/4 of the body of the barrier.

FIG. 3. Shows a superior elevation view of the tissue expansion barrier of this invention with integral pre-placed sutures and attached curved needles.

FIG. 4. Shows method of preparing incisions and placement of tissue barriers of this invention in incisions placed radially in ciliary sclera to produce circumferential expansion.

FIG. 5. Shows a cross-section view of the tissue barrier of this invention in expanded incision, sutured and covered by conjunctiva.

FIG. 6. Shows another embodiment of the invention, with “wings” projecting from each side of the lower edge of the barrier to aid in fixation of the device in the base of the expanded incision.

FIG. 7 Shows a further embodiment of the invention, with a trapezoidal cross section with the base wider than the top to aid in fixation and to prevent displacement.

FIG. 8. Illustrates the rationale of the invention, showing the effect of incisions in adding tissue.

FIG. 9. Shows the effect of radial incisions in the sclera over the ciliary body, expanding the globe.

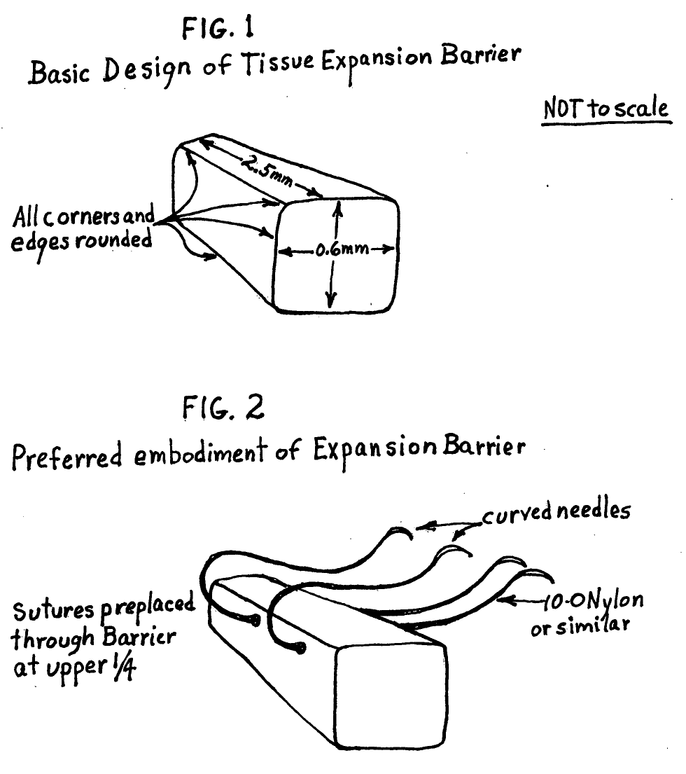

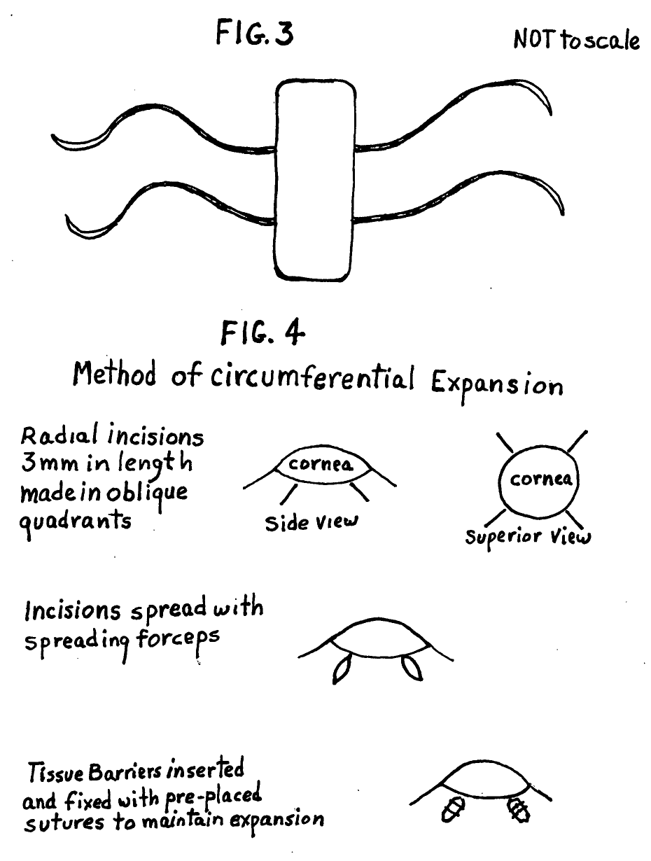

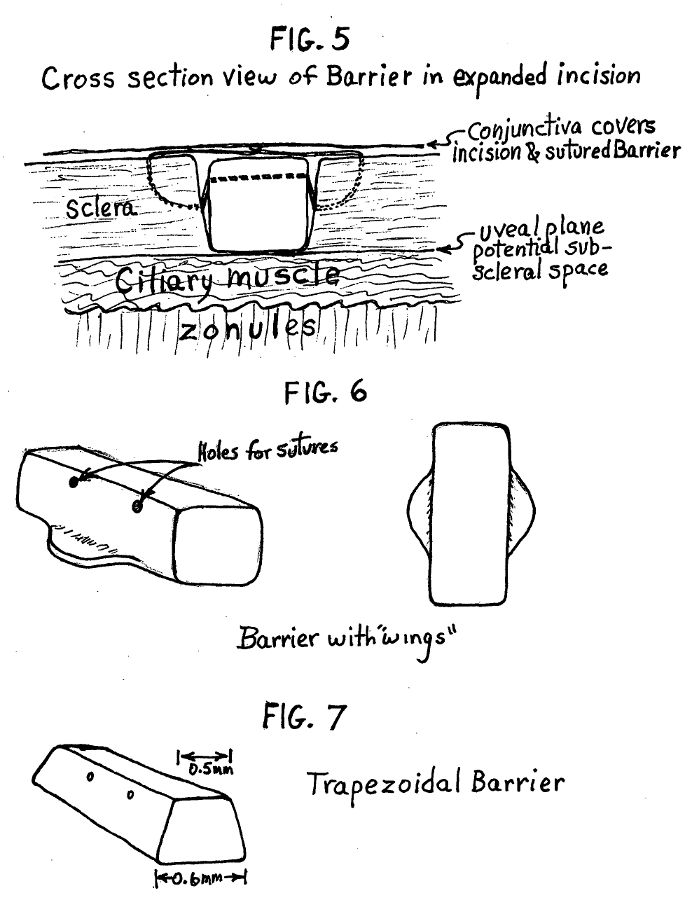

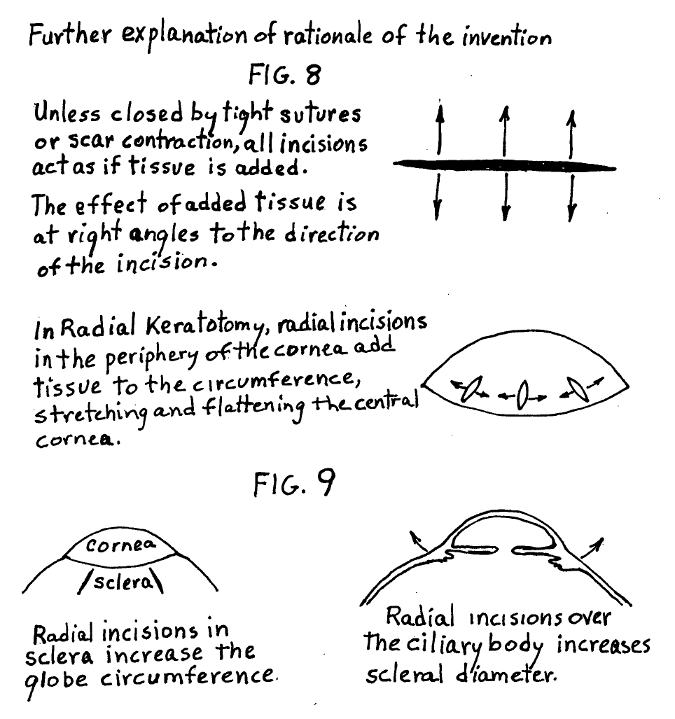

The invention having now been described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than the foregoing description, and all changes that come within the meaning and range of equivalency of the claims are intended to be embraced therein.

1. A method of increasing the amplitude of accommodation of a human eye having a crystalline lens and ciliary muscle and suspensory zonule fibers comprising increasing the effective working distance of the ciliary muscle from the lens by increasing the radial distance between the equator of the crystalline lens and the inner diameter of the ciliary muscle by enlarging the area called the posterior chamber through intervention with external means.

2. A method of treating presbyopia in a human eye having a crystalline lens and ciliary muscle and zonules connecting the ciliary muscle to the lens by increasing the effective working distance between the equator of the crystalline lens and the inner diameter of the ciliary muscle by increasing the radial distance between the equator of the crystalline lens and the inner diameter of the ciliary muscle by manipulating said muscle with intervention with external means.

3. A method of treating primary open angle glaucoma in a human eye having a crystalline lens and ciliary muscle and zonules connecting the ciliary muscle to the lens by increasing the effective working distance between the equator of the crystalline lens and the inner diameter of the ciliary muscle by increasing the radial distance between the equator of the crystalline lens and the inner diameter of the ciliary muscle by manipulating said muscle with intervention with external means.

4. A method of treating ocular hypertension in a human eye having a crystalline lens and ciliary muscle and zonules connecting the ciliary muscle to the lens by increasing the effective working distance between the equator of the crystalline lens and the inner diameter of the ciliary muscle by increasing the radial distance between the equator of the crystalline lens and the inner diameter of the ciliary muscle by manipulating said muscle by intervention with external means.

5. The method of claim 1 wherein said radial distance is increased by circumferentially expanding the sclera adjacent to the ciliary body.

6. The method of claim 1 wherein the scleral expansion is accomplished by making radial incisions in the sclera, extending about 3 mm from the limbus over the ciliary body.

7. The method of claim 6 wherein said scleral expansion is accomplished by stretching the radial incision.

8. The method of claim 7 wherein the stretching and expansion of the radial incision is maintained and preserved by insertion of a tissue barrier of the present invention into the incision.

9. The method of claim 8 wherein a tissue barrier comprising of an elongate or rectangular body that is inert and/or non reactive in tissue and adapted for insertion into an incision in the sclera of the eye to prevent tissue in-growth and maintain expansion.

10. The method of a tissue barrier as in claim 8 wherein the barrier is made of silicone, having the characteristics of being solid, flexible, inert, relatively soft, and similar in texture and appearance to the sclera.

11. The method of claim 8 wherein said barrier is approximately 2.5 mm in length and approximately 0.6 mm in diameter.

12. The method of a tissue barrier with one or more integral pre-placed sutures through the barrier approximately 1/4 the distance from the upper side.

13. The method of claim 8 wherein the said tissue barrier is fastened within the incision by pre-placed sutures integral to the barrier of the preferred embodiment of the invention.

14. The method of a tissue barrier of claim 8 wherein the barrier is made of a material such as silicone, titanium or polymethylmethacrylate or similar inert or non-reactive material.

15. The method of claim 8 wherein a barrier of the present invention is provided with pre-placed holes for sutures.

16. The method of claim 8 wherein the said tissue barrier is fastened by an adhesive.

17. The method of another embodiment of claim 9 wherein the tissue barrier is of a shape that aids in retention in an incision, such as in FIG. 6, with wings or projections extending from the sides or ends of the barrier.

18. The method of another embodiment of claim 9 wherein the tissue barrier is of a shape that aids in retention in an incision such as in FIG. 7 with a trapezoidal cross section.

19. Other configurations and dimensions can be used within the meaning and definition of the methods of the foregoing, and it is to be understood that even though numerous characteristics and advantages of the present invention have been set forth in the foregoing description, together with details of the structure and function of the invention, these are given by way of example only and changes may be made, particularly in matters of size and shape, within the spirit and scope of these claims.
